(19) United States
(12) Patent Application Publication
(10) Pub. No.: US 2011/0319862 A1
(43) Pub. Date: Dec. 29, 2011

(54) DELIVERY DEVICE AND METHOD

(75) Inventors: Steven A. FRIEDMAN, San Francisco, CA (US); Nicholas A. NELSON, San Francisco, CA (US)

(73) Assignee: Medavie Technologies, San Francisco, CA (US)

(21) Appl. No.: 13/169,982

(22) Filed: Jun. 27, 2011

Related U.S. Application Data
(60) Provisional application No. 61/398,356, filed on Jun. 25, 2010.

(51) Int. Cl. A61M 37/00

(52) U.S. Cl. 604/500; 604/131

(57) ABSTRACT

A device for automatically delivering a basal flow of a medication to a patient while also permitting manual control over bolus delivery of the medication is disclosed. The device is configured to permit bolus delivery to occur precedent, concurrent with, or subsequent to the basal delivery. A method for performing the same is also disclosed.

### CROSS-REFERENCE TO RELATED APPLICATION

This application claims the benefit of U.S. Provisional Application No. 61/398,356, filed Jun. 25, 2010, which is herein incorporated by reference in its entirety.

### BACKGROUND

1. Field of the Invention

This invention relates to devices and methods for delivering matter, such as fluids, for example an insulin solution, into an animal, and methods of use of the same.

2. Description of the Related Art

Diabetes is often treated by insulin delivery to a patient's tissues by injection to control blood sugar levels. The insulin is used to replace the deficiency of natural insulin creation or absorption by the patient's own body.

Significant variations in blood insulin levels due to factors such as dosing, diet, activity level and other physiological metrics, may cause drastic swings in the patient's need for insulin over time. For example, a slow steady basal dosing of insulin may be sufficient for most of a patient's day, but at times the patient may need a large dose of insulin. Patients may inject a bolus of insulin with a hypodermic needle when the need arises.

Accordingly, a need for a delivery pump that can deliver a basal and a bolus dose of insulin to a patient is desired. A device that can automatically control the basal dose yet have manual control over the bolus dose is also desired.

### SUMMARY OF THE INVENTION

A device for delivery of matter in or into a biological body is disclosed. A method for delivery of the same is also disclosed. The device can have a piston system that acts as a pressure control element configured to extract medication from a reservoir and dispense dosage into the patient. The device can have piston systems that can be manually driven, motor-driven, spring-driven, driven by shape changes in a shape-memory metal (e.g., Nitinol) wire, or any combination thereof. The reservoirs can be hard (e.g., a rigid cartridge) or soft (e.g., a bag or membrane). The device can be used to deliver single medications using different drivers for bolus and basal delivery rates.

A variation of the device can deliver a fluid that can be a solution containing insulin. The device can have a reservoir containing the fluid and having a fluid pressure. The device can have a basal control configured to change the fluid pressure in the reservoir. For example, the basal control can have an electric motor driving a worm gear and series of other gears. The device can have a bolus control configured to change the fluid pressure in the reservoir.

The device can have a pressure control element in fluid communication with the reservoir and animal tissue. The basal control can be configured to control the pressure control element, and the bolus control can be configured to control the pressure control element. The device can have a reservoir in fluid communication with the pressure control element. The device can have an outlet port in fluid communication with the pressure control element. The outlet port can be configured to connect directly or indirectly to a subcutaneous needle, to provide fluid communication from the reservoir through the pressure control element to the tissue. The device can have an inlet port that can allow fluid medication to be delivered from outside of the device to the reservoir.

The device can have a first valve, such as a reservoir valve, between the reservoir and the pressure control element. The device can have a second valve, such as an outlet valve, located inside the pressure control element or between the pressure control element and the outlet port or tissue. The first and second valves can be one-way valves directed in the same direction. The bolus and basal controls can be driven independent of each other to extract a dosage volume of fluid from the reservoir. The pressure control element can be spring-driven to deliver the extracted dosage volume through the outlet port and into the tissue. The basal control can counteract the force of the spring driving the pressure control element. The bolus control can counteract the force of the spring driving the pressure control element. The bolus control can have a handle and/or button, and be exclusively manually operated. The basal control can have an electric control to extract a dosage volume from the reservoir. The basal control can be electro-mechanical. The electric basal control can have a motor and a worm gear. The bolus control element can operate independently of the basal control element.

The pressure control element can have a piston. The piston can be spring-loaded. The piston can have a piston fluid channel along the entire length of the piston. The piston fluid channel can be an open fluid channel that can allow fluid to pass through the piston from the holding cylinder or chamber, through the second valve and out the outflow port and to tissue. The reservoir can be flexible.

The limit of translational motion of the piston can be adjusted by adjusting a piston control such as a dial. The dial can be mechanically attached to the piston by a rack (e.g., on the piston) and pinion (e.g., the axle of the dial). Adjustment of the dial can limit the eventual translational motion of the piston within the holding cylinder or chamber. Controlling the motion of the piston can control or affect the volume of the fluid delivered in a stroke of the piston assembly, such as during basal or bolus delivery. The reservoir can be flexible or rigid. The reservoir can be delivered to the user pre-filled with fluid or can be delivered to the user empty. The user can fill or refill the reservoir with fluid.

The inlet of an internal outlet connector, such as tubing, or any other flexible or rigid flow channel, can be attached to the outlet of the piston fluid channel. The outlet of the internal outlet connector can be attached to the outlet port of the device.

The outlet port of the device can be attached to a tubing and needle assembly, such as an infusion set, extending externally from the device and attaching remotely to the patient, such as when the device is worn on the patient with or in a belt or strap. The outlet port of the device can be attached directly to a needle, catheter, or any other subcutaneous flow channel, such as when the outlet port of the piston is configured for a body-worn embodiment of the device. An iontophoresis or electroporation device can be attached to the outlet port or to tubing or another channel connected to the outlet port.

The device can have an adhesive surface configured to attach to the skin of a patient. For example, the bottom surface of the device can be partially or completely covered with a dermatologically safe adhesive.

A device for delivering a fluid to a biological tissue is also disclosed that has a flexible reservoir containing the fluid and having a fluid pressure, a spring-loaded pressure control element in fluid communication with the reservoir, an electric control configured to control the pressure control element, such as by moving the pressure control element against the spring, and a manual control configured to control the pressure control element such as by moving the pressure control element against the spring. The manual control element can override the electric control element. The pressure control element controllably alters the pressure in the reservoir, for example by moving the pressure control element.

Also disclosed is a method for delivering a fluid to a biological tissue. The method includes delivering a basal infusion of the fluid from a reservoir of a fluid delivery device. The basal infusion includes changing the pressure of the reservoir with a pressure control element controlled by an electric motor. The method also includes delivering a bolus of the fluid from the fluid delivery device. The bolus fluid delivery includes changing the pressure of the reservoir with a pressure control element, for example, controlled by a manual input force from a user. The bolus fluid delivery and/or the basal infusion can include delivering the fluid to the tissue under positive pressure generated by a spring force applied against a piston. The spring force can be generated by the same spring for the basal and the bolus delivery. The piston can be the same piston for the basal and the bolus delivery.

The method can include stopping the delivering of the basal infusion of the fluid before delivering the bolus of the fluid. The delivering of the basal infusion of the fluid and/or the delivering of the bolus of the fluid can deform the reservoir.

The method can include attaching the fluid delivery device to a skin surface of a patient, for example with a strap, harness, tape, an adhesive surface of the device, or combinations thereof.

The delivering of the basal infusion can include delivering fluid continuously for an extended period of time defined by the type of medication or other agents within the fluid. The delivering of the bolus can include delivering fluid for single doses over short periods of time defined by the type of medication or other agents within the fluid. For example, for delivery of insulin, basal delivery would be from about 0.1 Units per hour (e.g., 0.001 mL/hour of insulin) to about 4 Units per hour (e.g., 0.04 mL/hour of insulin).

### BRIEF DESCRIPTION OF THE DRAWINGS

FIGS. 1a through 1c are side, top and bottom views, respectively, of a variation of the device.

FIG. 1d is a top, partially see-through view of the variation of the device of FIGS. 1a and 1b in a configuration before loading or after release of the piston spring.

FIG. 1e is a top, partially see-through view of the variation of the device of FIG. 1d in a configuration where the piston spring is loaded and the piston is retracted by the basal delivery mechanism.

FIG. 1f is a top, partially see-through view of the variation of the device of FIG. 1d in a configuration where the piston spring is loaded and the piston is retracted by the bolus delivery mechanism.

FIG. 2a illustrates a variation of the piston assembly, internal outlet connector, and needle.

FIG. 2b is a cross-sectional view of the piston assembly, internal outlet connector and needle of FIG. 2a.

FIG. 3 is a top, partially see-through view of a variation of the device.

FIG. 4 illustrates a method of constructing a variation of the device by inserting a power pack into a pump.

FIG. 5 illustrates three variations of the power pack.

FIG. 6 is a close-up side perspective view of a method of removing a variation of the power pack from FIG. 5 from the pump of FIG. 4.

FIG. 7 illustrates a method of controlling the device constructed in FIG. 4.

FIGS. 8a through 8c illustrate a variation of the bolus delivery mechanism for the device in different configurations.

FIGS. 9a and 9b illustrate a variation of the device having a basal delivery mechanism with a power pack in different configurations.

FIG. 10 illustrates a variation of the delivery mechanism of the device.

FIG. 11 is a schematic figure of a method or operation of the mechanism shown in FIG. 10.

FIGS. 12a through 12g illustrate variations of methods for using the device.

FIGS. 13a and 13b are perspective and top views of a variation of the device.

### DETAILED DESCRIPTION

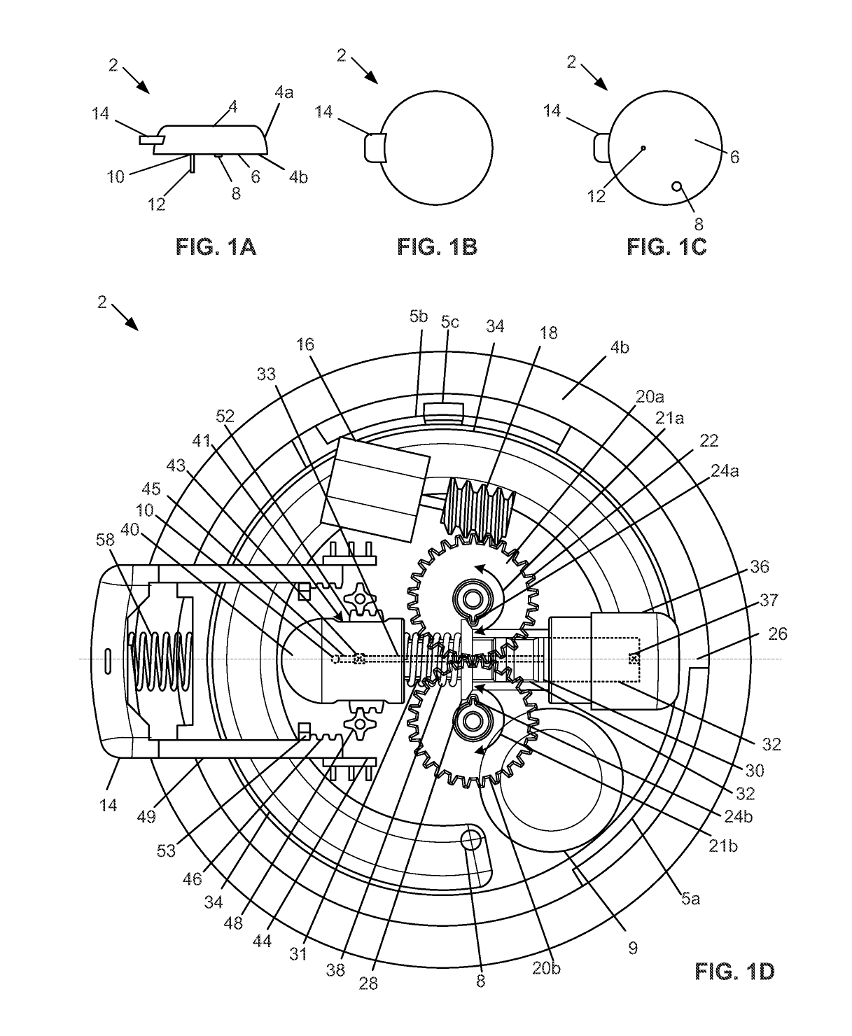

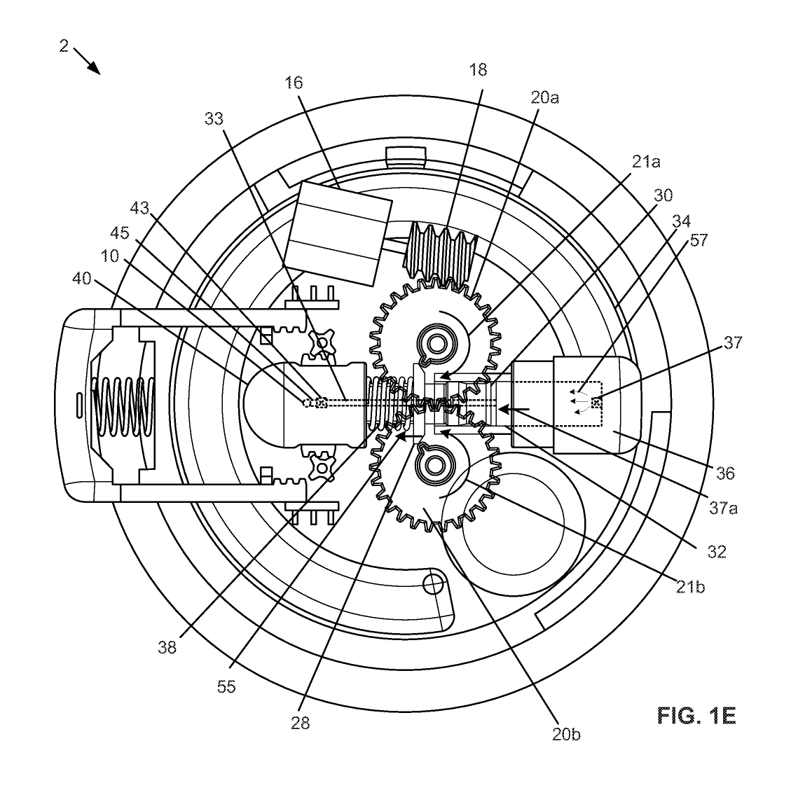

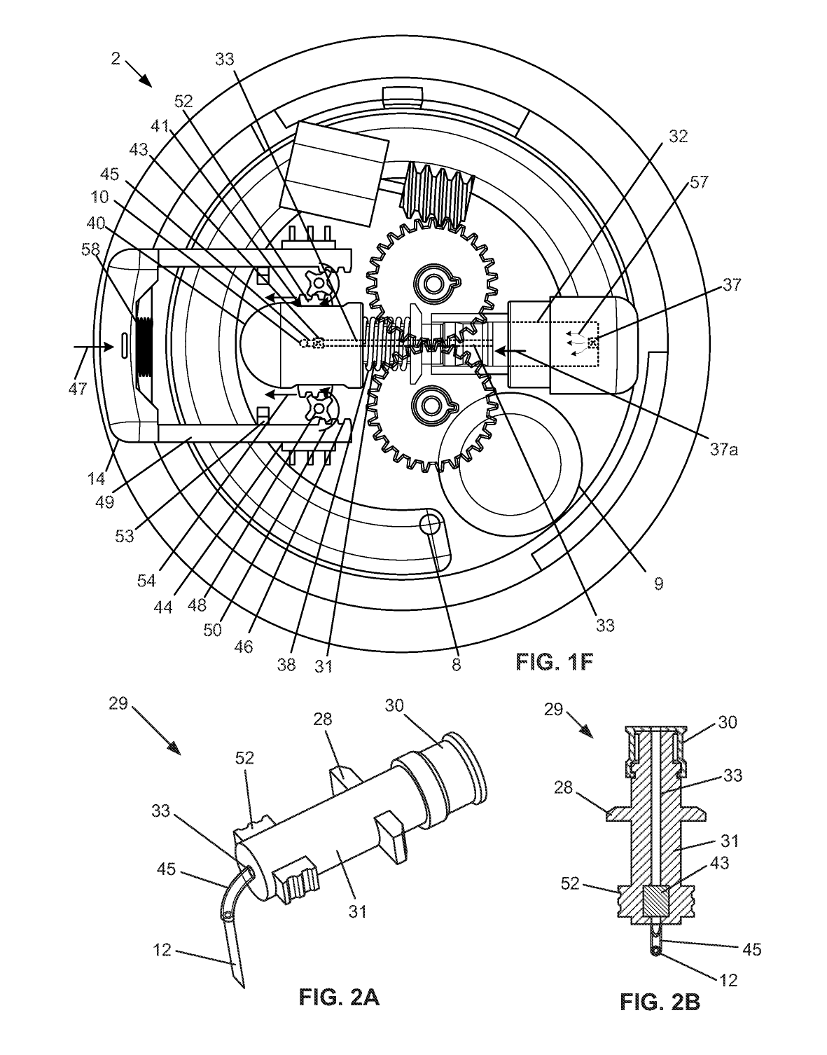

FIGS. 1a through 1f illustrate a device 2 configured to deliver matter to biological tissue. The device 2 can be used to deliver liquids, gels, gases, solids or combinations thereof subcutaneously, transcutaneously, viscerally or combinations thereof. For example, the device can be used to deliver an insulin solution to a human patient for treatment of diabetes transcutaneously, for example, intravenously.

The device 2 can be configured to deliver a basal dose of flowable matter and/or a bolus dose of flowable matter. For example, the basal dosing can be continuous, constant or cycling delivery of matter, such as a flow of a liquid, over about one minute, more narrowly over about five minutes. The basal dosing can be continuous for hours, such as about 3 hours, about 5 hours, about 8 hours or more. The bolus dosing can be a delivery of matter one-time under about one minute, more narrowly under about 30 seconds. The device 2 can be configured to deliver the basal dosing automatically. The device 2 can be configured to deliver the bolus dosing manually. The basal and bolus dosing can be delivered from the same or different reservoirs.

The device 2 can have a case 4. The case 4 can be a hard (i.e., rigid) or soft (i.e., flexible) case. The case 4 can have a case top 4a and a case bottom 4b. The case top 4a can be removable from the case bottom 4b, for example allowing access to the internal volume of the case to replace the battery or reservoir. The durable and consumable components can be housed in separate casings and/or within sub-compartments of a single case 4. The case top 4a can removably attach to the case bottom 4b with angular tongues 5a, grooves 5b, and snap-fit components 5c. The base bottom 4b can have a bottom surface 6. The case bottom surface 6 can be flat (i.e., planar) or curved to fit an anatomical feature, such as an organ or skin surface. The case bottom surface 6 can have an attachment coating, such as an adhesive. The case bottom surface 6 can be attached to a patient's skin or organ surface. The case bottom surface 6 can be attached to one or more articles of clothing or secured inside a protective case that is attached to clothing.

The surface of the case 4 can have an intake port 8, such as an open port or a covered port. The intake port 8 can be covered by an openable panel or self-sealing silicone or rubber membrane. The intake port 8 can be flush with the top or bottom surface of the remainder of the case 4 or can be raised or sunken compared with the surrounding surface of the case 4. The intake port 8 can be self-sealing. For example, a needle can be inserted through the membrane, the flowable material to be delivered to the patient can be injected through the needle into an internal reservoir and through the fluid channels of the device 2, and the needle can be removed from the membrane. The membrane can then substantially close and seal the puncture through which the needle passed through the membrane. The intake port 8 can be configured to receive one or more ampoules, carpoules, cartridges or reservoirs that are pre-filled with the matter to be delivered to the patient.

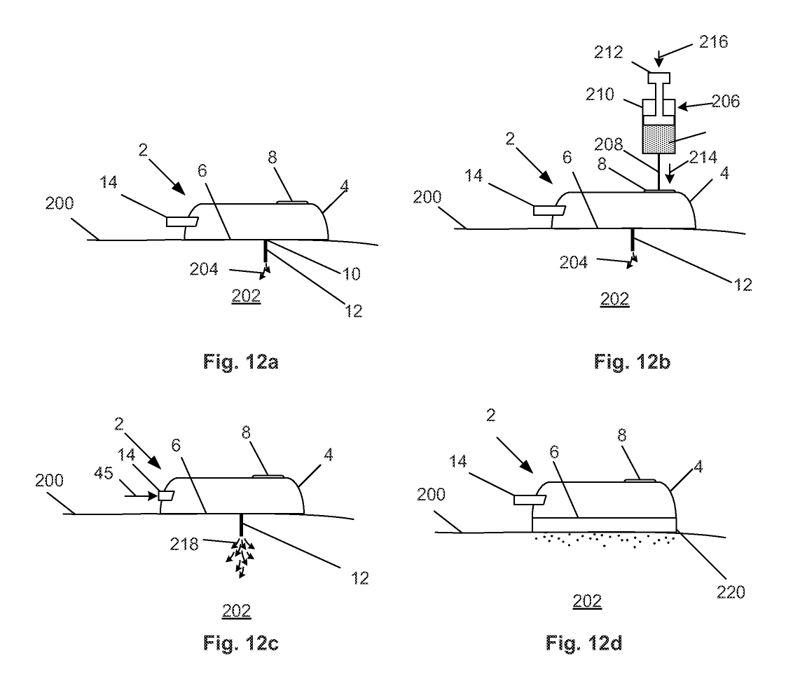

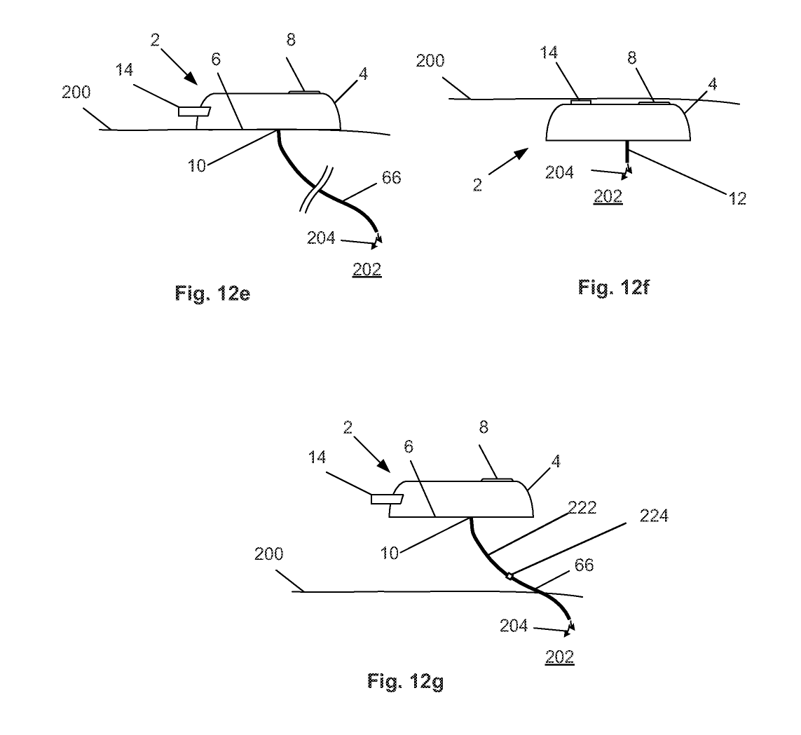

The case 4 can have one or more outlet ports 10. The outlet ports 10 can be on the case bottom surface 6. The flowable matter to be delivered to the patient can be delivered through the outlet port 10. The outlet port 10 can be substantially always open or unobstructed or closeable. The outlet port 10 can be attached to a distal delivery element, such as outlet needle 12, that can deliver the matter to a remote location away from the outlet port 10. Other distal delivery elements are described infra, and shown in FIGS. 12A-12E.

The case 4 can have a bolus control element, such as including a bolus button 14, activatable from outside of the case. The bolus button 14 can extend through a hole in the case 4. The bolus button 14 can extend from the lateral side of the case 4, for example, away from the intake port 8 and the outlet port 10. The bolus button 14 can be configured to be depressed by a user exerting a force directed toward the case 4. Pressing the bolus button 14 can control the device 2 to deliver a bolus of flowable material through the outlet port 10. Part or all of the bolus control element can be removed from the device 2, for example, without impairing the performance of the basal control element. The bolus control element can include other components described infra, such as a bolus spring, bolus button racks, pinions, sliders, and piston racks.

FIG. 1d illustrates that the device 2 can have an electronics master component 9. The electronics master component 9 can have a power source, such as a battery or external power supply, and a control board (e.g., having a microprocessor). The electronics master component 9 can be in wired or wireless power and/or data communication with the motor, the valves, position or motion sensors on the pinion gears, a volume and/or pressure sensor on the reservoir, or combinations thereof.

The device 2 can have a basal control element. Part or all of the basal control element can be removed from the device 2, for example, without impairing the performance of the bolus control element. The basal control element can include components described infra, such as a motor, a worm gear, and drive gears, or a basal power pack, or a shape memory wire, a piston cuff attached to the shape memory wire, and an electronics board, or combinations thereof.

The basal control element can include a motor 16. The motor 16 can be a The motor 16 can be one or more brushed DC motors, brushless DC motors, coreless DC motors, stepper motors, and nanoelectromechanical systems (NEMS), spring-loaded solenoids, or combinations thereof. As one non-binding example, the motor 16 can be a 32 g, 2.4V precious metal-brush motor that runs most efficiently at 6850 RMP, 0.82 A, with a torque at 18.6 g(cm). The motor 16 can be controlled by a motor controller or the control board of the electronics master component 9. The motor 16 can be directly mechanically connected, or connected through a transmission, to a worm gear 18. The worm gear 18 can transmit torque and power from the motor 16, from a rotation along a first axis, to a first drive gear 20a that can be configured to rotate about a second axis perpendicular to the first axis, as shown by arrow 21a.

The gear teeth 22 on the first drive gear 20a can engage gear teeth 22 on the second drive gear 20b. The first drive gear 20a can transmit torque to the second device gear 20b, rotating the second drive gear 20b, as shown by arrow 21b. The first drive gear 20a can each have one or more first control teeth 24a. The second drive gear 20b can have one or more second control teeth 24b. The first control teeth 24a can be symmetrically positioned across an axis of symmetry 26 with respect to the second control teeth 24b.

Any or all of the control teeth 24 can engage a piston cuff 28. The piston cuff 28 can be part of a piston assembly 29. The piston or piston assembly 29 can have an elastomeric (e.g., rubber or silicone) piston seal 30 having a fluid-tight seal within a cylinder 32. The piston seal 30 can be in fluid communication with a reservoir 34 via a reservoir connector 36. The piston assembly 29 can have a piston rod 31. The piston assembly 29 can have one, two or more piston racks 52 located on opposite sides of the piston rod 31. The piston assembly 29 can have a piston fluid channel 33 that can extend through the piston rod 31 and piston seal 30 along the entire length of the piston assembly 29. The elements of the piston assembly 29 can be integral or fixedly attached to each other. The piston assembly 29 can translatably slide within the cylinder 32

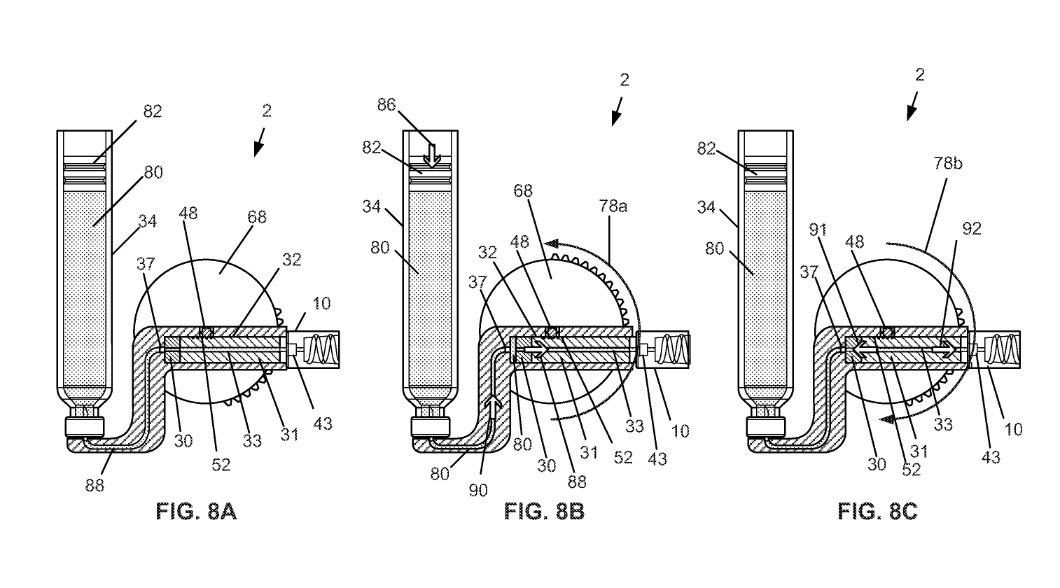

The reservoir 34 can be filled with a flowable material to be delivered to the patient. The reservoir 34 can be refilled through the intake port 8. The reservoir 34 can be entirely or partially within the case 4. The reservoir 34 can have a soft, flexible bladder. The reservoir 34 can be vented (e.g., when rigid) or have a variable volume (e.g., as shown in FIGS. 7a-8b). The reservoir 34 can have one or more ampoules, carpoules, cartridges or combinations thereof that can be removed and replaced.

A reservoir connector 36 can be fixed to the case 4. The reservoir connector 36 can be in fluid communication with the reservoir 34 and the cylinder 32. The reservoir connector 36 can have a one-way reservoir valve 37 in fluid communication with the reservoir 34 and the cylinder 32. The reservoir valve 37 can allow flow in the direction from the reservoir 34 to the cylinder 32. The reservoir valve 37 can prevent flow in the direction from the cylinder 32 to the reservoir 34. The cylinder 32 can extend into the reservoir connector 36.

A piston coil spring 38 can be positioned around the piston rod 31. The piston spring 38 can be constrained between the piston cuff 28 and a housing 40.

The housing 40 can be fixed to the case 4, such as to the case bottom 4b. The end of the piston assembly 29 can slide within the housing 40. The piston racks 52 can extend laterally out of the housing 40 through housing lateral ports 41. The housing lateral ports 41 can be long enough to allow the piston racks 52 to slide without being interference fit against the end of the housing lateral ports 41 for all of the range of motion for the piston assembly 29.

The piston fluid channel 33 can have an outlet valve 43. The outlet valve 43 can be in, in line with, or at the end of the piston fluid channel 33, for example within the volume defined by the perimeter of the housing 40. The outlet valve 43 can be located inside the piston rod 31, for example toward the outlet end of the piston rod 31. The outlet valve 43 and/or the outlet end of the piston fluid channel 33 can attach to and be in fluid communication with the internal outlet connector 45. The internal outlet connector 45 can attach to and be in fluid communication with the outlet port 10. The outlet valve 43 can be a one way valve. The outlet valve 43 can allow fluid flow in the direction from the piston fluid channel 33 to the internal outlet connector 45 and the outlet port 10. The outlet valve 43 can prevent flow in the direction from the internal outlet connector 45 and the outlet port 10 to the piston fluid channel 33.

The internal outlet connector 45 can be a flexible tube. For example, the internal outlet connector 45 can flex and bend to allow the movement of the piston assembly 29 during use without significant resistance and maintaining connections with the piston fluid channel 33 and the outlet port 10. The internal outlet connector 45 can maintain a substantially constant volume during use.

The bolus button legs 49 can translatably slide along bolus button sliders 44 fixed to the case 4. The sliders 44 can restrain the motion of the bolus button 14 to one-dimensional translation along the axis of symmetry 26.

The bolus button 14 can have bolus button legs 49. The bolus button legs 49 can have one or more button racks 46. The button racks 46 can have teeth configured to engage with the teeth on the pinion gears 48. The button racks 46 can be at or near the terminal ends of the bolus button legs 49.

The case 4 can be fixed to button stops 53. The button stops 53 can interference fit against the end of the button racks 46. The button stops 53 can limit the retraction of the bolus button 14 from the case 4.

A bolus coil spring 58 can be constrained between the bolus button 14 and the case 4. The bolus spring 58 can be in compression.

FIG. 1e illustrates that the motor 16 can drive the worm gear 18, which can drive the first drive gear 20a. The first drive gear 20a can drive the second drive gear 20b. The control teeth 24a and 24b of the first and second drive gears 20a and 20b can pull, as shown by arrow 55, the piston cuff 28 away from the reservoir connector 36. The piston cuff 28 is fixed to the piston seal 30 and translates, as shown by arrow 57, the piston seal 30 with the piston cuff 28. As the piston seal 30 translates, as shown by arrow 37a, away from the reservoir connector 36, the piston seal 30 can create a negative pressure in the rigid and vented, or soft, reservoir 34, drawing flowable contents from the reservoir 34 through the reservoir valve 37, as shown by arrows 57, and into the cylinder 32. The outlet valve 43 can prevent flowable material from flowing from the side of the outlet valve 43 away from the cylinder 32 to the cylinder 32.

As the piston seal 30 retracts from the reservoir connector 36, the piston cuff 28 can compress the piston spring 38 against the housing 40. The piston spring 38 can have a spring coefficient from about 0.01 lbs/inch to about 20 lbs/inch, more narrowly from about 0.05 lbs/inch to about 12 lbs/inch, yet more narrowly from about 0.08 lbs/inch to about 9.99 lbs/inch. The piston spring 38 can be constrained between the housing 40 and the piston cuff 28.

When the control teeth 24a and 24b rotate away from and disengage the piston cuff 28, the piston spring 38, in a compressed state, can deliver the spring force stored in the piston spring 38 to the piston cuff 28. The piston cuff 28 can translate the piston 30 toward the reservoir connector 36.

The positive pressure created within the cylinder 32 by the piston seal 30 translating toward the reservoir connector 36 can be sufficient to trigger the reservoir valve 37 to close and the flowable material in the cylinder 32 can be forced under pressure down the piston fluid channel 33. The outlet valve 43 can open. The positive pressure flow from the cylinder 32 can direct the flowable material through the outlet valve 43, through the internal outlet connector 45, and out of the outlet port 10. The reservoir valve 37 can prevent flowable material on the side of the reservoir valve 37 facing away from the reservoir 34 from flowing to to the reservoir 34.

The drive gears 20a and 20b can rotate so that the control teeth 24 rotate around the rotational axis of the respective drive gears 20a and 20b and re-engage the piston cuff 28. The above cycle can then automatically repeat as long as the motor 16 is rotating and engaged to the gears 18, 21a and 21b. The device can deliver desired basal dosage volumes of the material stored in the reservoir 34 to the outlet port 10 at a desired frequency for an extended period. For example the basal delivery can occur at any of the times described herein, for example, longer than one minute, longer than about five minutes, about six hours or until the motor is disconnected or disconnected from the gears, turned off, the power supply to the motor expires or combinations thereof.

The frequency of basal dosing can be from about 2 cycles per second to about 0.01 cycles per second, yet more narrowly from about 1.5 cycles per second to about 0.1 cycles per second, for example about 1 cycle per second. The delivery rate during basal delivery can be from about 0.25 U (e.g., 0.0025 mL of insulin) per hour to about 6 U (e.g., 0.06 mL of insulin) per hour of insulin, for example about 1 U (e.g., 0.01 mL of insulin) per hour.

FIG. 1f illustrates that when the bolus button 14 is manually pressed and translated, as shown by arrow 47, into the case 4, the button racks 46 can press into pinion gears 48 causing the pinion gears 48 to rotate, as shown by arrows 50. The button racks 46 can be engaged to or disengaged from the pinion gears 48 before the bolus button 14 is pressed. The bolus button 14 can compress the bolus spring 58 between the bolus button 14 and the case 4. The bolus spring 58 can have a spring coefficient (“K”) from about 0.01 lbs/inch to about 10 lbs/inch, more narrowly from about 0.08 lbs/inch to about 4.99 lbs/inch. The pinion gears 48 can drive piston racks 52 extending from the piston rod 31 outside of the housing 40. The pinion gears 48 can force the piston racks 52 to translate, as shown by arrow 54, away from the reservoir connector 36. The piston racks 52 can be fixed to the piston assembly 29, e.g., the piston rod 31, piston cuff 28 and the piston seal 30. The translation of the piston racks 52 can translate the elements of the piston assembly 29.

As the piston seal 30 translates away from the reservoir connector 36, as shown by arrow 37a, the piston seal 30 can create a negative pressure in the cylinder 32, which can draw flowable material, as shown by arrows 57, from the reservoir 34 through the reservoir valve 37 and into the cylinder 32, as described supra. The outlet valve 43 can prevent fluid from flowing from outside of the cylinder 32 to inside of the cylinder 32 through the outlet valve 43.

The bolus button 14 can reach a hard stop at maximum depression, such as against the fully compressed bolus spring 58, and/or the case 4. The hard stop indicates the maximum amount of additional flowable material that can be delivered from the reservoir 34 into the cylinder 32 in one piston stroke. After the bolus button 14 forces the retraction of piston assembly 29, as shown by arrow 54, the bolus button 14 can be released or manually retracted in a direction away from the case 4. The bolus button 14 can be returned to an undepressed or extended configuration due to pressure from the bolus spring 58 and/or the piston spring 38. The bolus spring 58 can be constrained and compressed between the bolus button 14 and the case 4 when the bolus button 14 is depressed.

As the bolus button 14 returns to an undepressed or extended configuration, the motion of the piston assembly 29 is reversed, in the reverse of arrows 54 and 37a. The piston assembly 29 can translate toward the reservoir connector 36. The piston seal 30 can translate toward the reservoir connector 36, triggering the reservoir valve to close and the pressure in the cylinder 32 to increase. The outlet valve 43 can then open, and the flowable material in the cylinder 32 can flow through the piston assembly 29, including through the piston fluid channel 33, through the outflow valve 43, through the internal outflow connector 45, and out of the outlet port 10. The flowable material in the cylinder 32 is then delivered to patient tissue (e.g., intravenously or intramuscularly) through the outlet port 10 as a single bolus delivery.

The bolus button 14 can then be in the undepressed or extended configuration and be used again repeatedly as needed.

The motor 16 can remain running or be turned off (automatically by a switch activated by pressing the bolus button 14, or manually) prior to pressing the bolus button 14. The bolus delivery can occur precedent to, concurrent with, subsequent to, or combinations thereof, the basal delivery. The bolus doses can be from about 1 U to about 6 U.

When the bolus button 14 is not depressed, the gap between the bolus racks 46 and the pinion gear 48 can be sufficient to eliminate contact between the bolus racks 46 and the pinion gear 48 during basal delivery operation of the device. When engaged with the piston cuff 28, the control teeth 24a and 24b can be positioned on the side of the piston cuff 28 facing the reservoir connector 36, for example, to prevent interfering with movement of the piston during bolus delivery. The motor 16 can be turned off and/or disengaged during bolus delivery, for example, to prevent resisting force from the control teeth 24a and 24b during movement of the piston cuff 28 during bolus delivery.

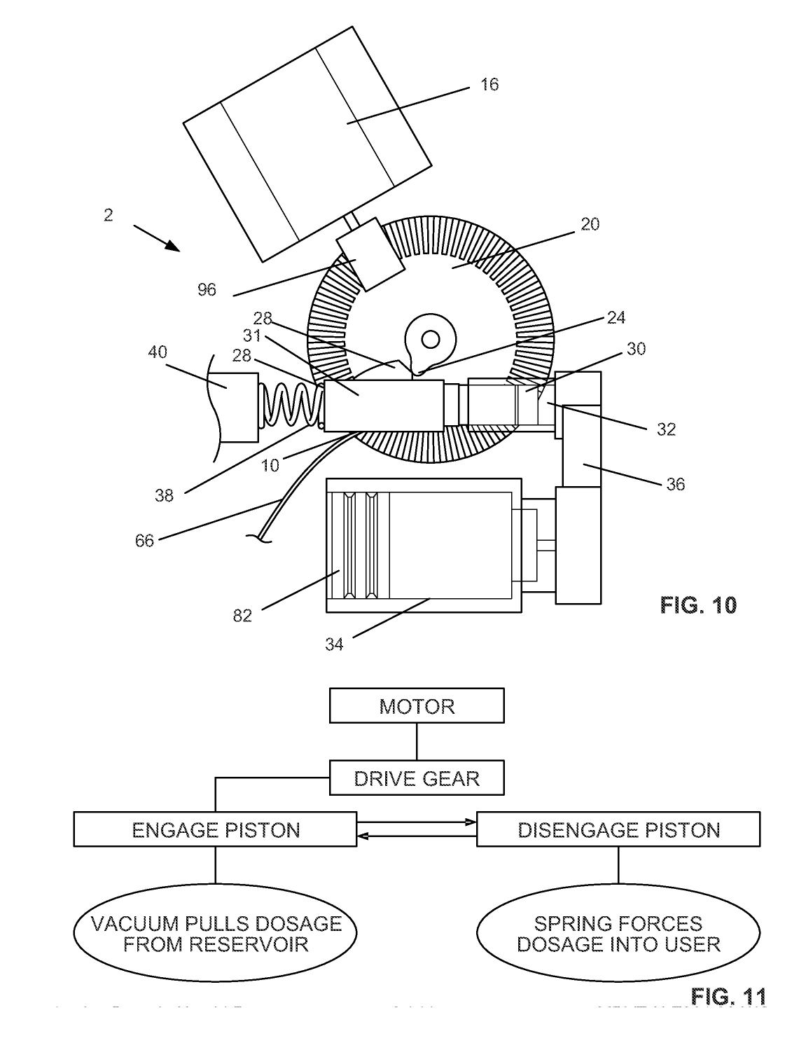

The bolus delivery volume (as shown in FIG. 10 can be significantly larger than the basal delivery volume (as shown in FIG. 1e), but are shown as comparable volumes in the figures for illustrative purposes.

The aforementioned elements can be fixed or rotationally attached directly or indirectly to the case 4.

FIGS. 2a and 2b illustrate that the piston assembly 29 can have a piston rod 31, a piston seal 30, one or more piston cuffs 28, one or more piston handles or racks 52, a piston fluid channel 33, and an outlet valve 43. An internal outflow connector 45 can be attached to the end of the piston fluid channel 33 exiting the piston rod (i.e., as opposed to exiting the piston seal 30). The internal outflow connector 45 can terminate at the outflow port 10, inside the case 4 or outside the case 4. The needle 12 can be attached to the terminal end of the internal outflow connector 45 not attached to the piston rod 31. The internal outlet connector 45 can be a flexible tube. For example, the internal outlet connector 45 can flex and bend to allow the movement of the piston assembly 29 during use without significant resistance and maintaining connections with the piston fluid channel 33 and the outlet port 10. The internal outlet connector 45 can maintain a substantially constant volume during use.

The piston cuff 28 can extend radially from the piston rod 31. The piston cuff 28 can encompass the circumference of the piston rod 31 or can extend in discrete increments (as shown in FIG. 2a) around the circumference of the piston rod 31.

FIG. 2b illustrates that the outlet valve 43 can be positioned within the piston assembly 29, for example in the piston rod 31. The outlet valve 43 can be positioned internally adjacent to the piston rack 52. The outlet valve 43 can be at one end or at an intermediate location along the length of the piston fluid channel 33.

The piston seal 30 can coat or wrap around the terminal end of the piston rod 31 proximal to the reservoir connector 36.

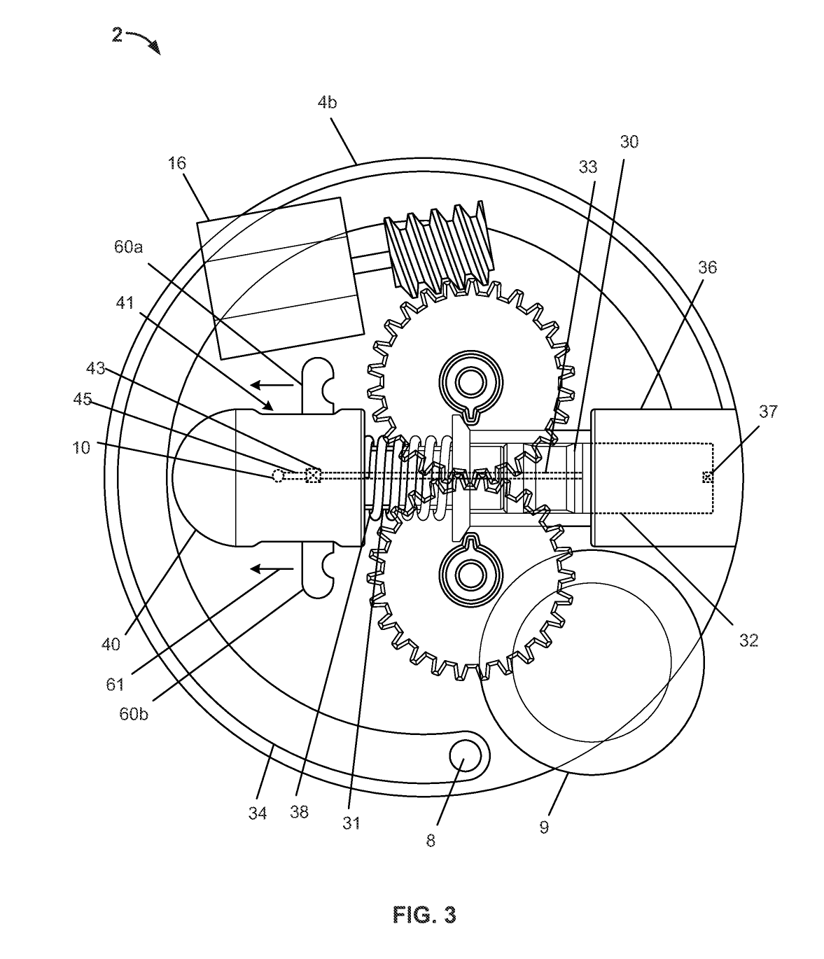

FIG. 3 illustrates that the piston assembly 29 can have a piston first handle 60a and a piston second handle 60b extending laterally from the piston rod 31. The piston handles 60a and 60b can extend laterally out of the housing 40 through housing lateral ports 41. The housing lateral ports 41 can be long enough to allow the piston handles 60a and 60b to slide longitudinally through the housing lateral ports 41 without being interference fit against the end of the housing lateral ports 41 for all of the range of motion for the piston assembly 29.

The drive gears 20a and 20b can be disengaged manually or automatically from the piston cuff 28 before or concurrent with manual activation of the handles 60a and 60b. The drive gears 20a and 20b can be have clutches that can activate to disengage and/or the drive gears 20a and 20b can be translated out of plane with the piston cuff 28, for example, driven by a link attached to the housing 40. The electronics master component 9 can stop the drive gears 20a and 20b at the positions shown in FIGS. 1d and 3 (i.e., touching, but not pressing against the piston cuff 28) when the electronics master component 9 detects that the handles 60a and 60b (or the bolus button 14 for other variations) have been moved.

The user can pull on the handles 60a and 60b, as shown by arrows 61. The handles 60 can be linked to an ergonomic gripping feature exposed outside case 4 such that the user can apply a pulling force to the piston assembly 29 to translate the piston assembly 29, including the handles 60a and 60b, piston rod 31 and piston seal 30, and outlet valve 43, away from the reservoir connector 36. The piston seal 31 can generate a negative pressure in the cylinder 32, drawing in flowable material from the reservoir 34, as shown in the cylinder in FIG. 1f. The handles 60a and 60b can then be released and the device can deliver a bolus of the flowable material through the outlet port (as described supra).

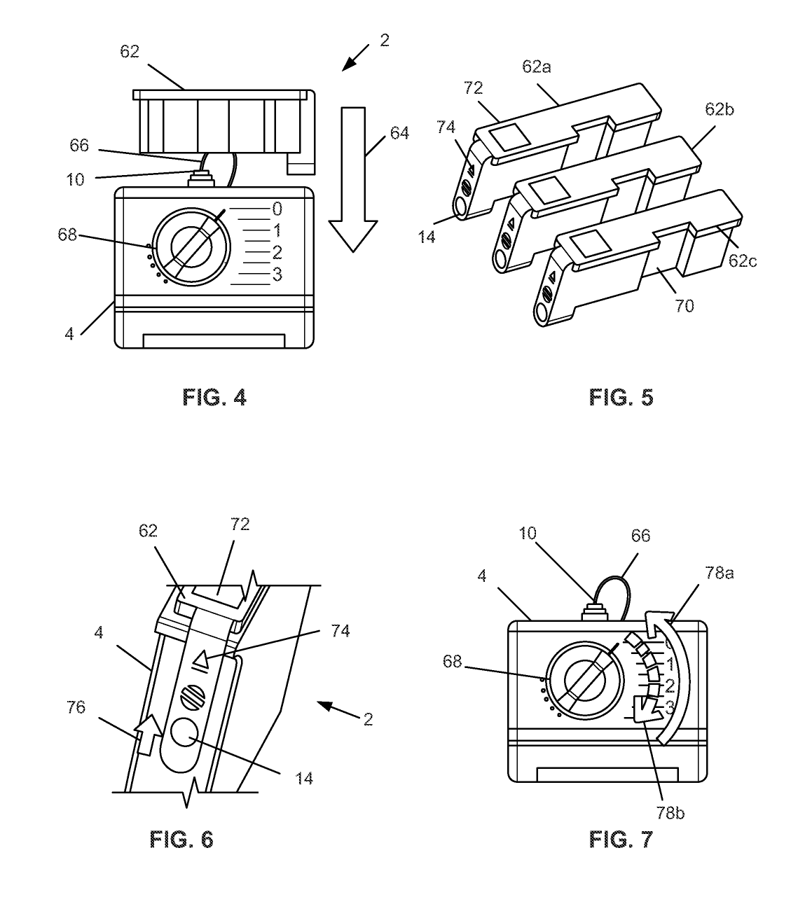

FIG. 4 illustrates that the case 4 can be configured to receive a basal power pack 62. The basal power pack 62 can have a power supply, the motor 16, the reservoir 34, the flowable material, such as an insulin solution, the piston assembly 29, cylinder 32, any of the other elements shown and described in FIGS. 1a-3, or combinations thereof. The power pack 62 can be inserted, as shown by arrow 64, into or onto the case 4. The case can have a delivery catheter 66 attached to and in fluid communication with the outflow port 10. The case 4 can have a bolus dial or bolus quantity delivery control 68. The bolus quantity delivery control 68 can be a dial. The bolus quantity delivery control 68 can change the volume of bolus delivered in each bolus delivery. Basal delivery of the flowable material can begin when the basal power pack 62 is inserted into the case 4, or when the case 4 is turned on and/or activated. The bolus delivery quantity can be controlled by the dial 68.

FIG. 5 illustrates a variety of basal power packs 62a, 62b, and 62c including power packs with fixed basal delivery rates and power packs with adjustable basal delivery rates. The various basal power packs 62a, 62b, and 62c can have similar or identical form factors, for example, to fit the same case 4. For example, the basal power packs 62a, 62b, and 62c can have a unidirectional groove 70. The groove 70 can fit the case 4 and prevent the basal power pack from being inserted in the wrong orientation into the case 4. The basal power packs 62 can have labels or displays 72. The displays 72 can indicate the power, total energy stored, type of flowable material contents, settings for flow rate and frequency, alerts for flow blockage, or combinations thereof. The basal power packs 62 can have a bolus button 14 (which can also or alternatively be located on the case 4 as dial 68). The basal power packs 62 can have a release button 74 (which can also or alternatively be located on the case 4). The release button 74 can detach the basal power pack 62 from the case 4 so the basal power pack 62 can then be removed from the case 4, and potentially replaced. The case 4 can hold more than one power pack 62 simultaneously.

FIG. 6 illustrates that the release button 74 can be depressed, and the power pack 62 can be slidably removed, as shown by arrow 76, from the case 4. The power pack 62 can be removed from the case 4 when the power is off.

FIG. 7 illustrates that the bolus quantity can be increased or decreased by rotating the dial, shown by arrows 78a and 78b. The bolus delivery can be triggered by rotating the bolus dial 68 to a set dosage (i.e., pulls the dosage), and then returning dial 68 to its original position (i.e., delivering the dosage). The bolus delivery can be operated without the basal power pack 62 inserted into the case 4. The bolus quantity delivery control 68 can be reset (e.g., rotated back to zero) when the basal power pack is reinserted into the case 4, for example, to resume basal delivery.

FIGS. 8a through 8c illustrate that the device 2 can have a reservoir 34 filled with flowable material 80, such as an insulin solution or agent. The reservoir 34 can be soft and flexible or hard and rigid, such as a removably attached cartridge, as shown. The reservoir 34 can be sealed with a reservoir piston 82. The reservoir piston 82 can be configured to slide within the reservoir 34 concurrent with sealing the reservoir 34. The flowable material 80 can flow along a path from the reservoir 34, through a reservoir valve 37, and into the cylinder 32. The flowable material 80 can then be pressurized by the piston seal 30 and exit the cylinder 32 through the piston fluid channel 33, the outlet valve 43, and out the outlet port 10.

As the flowable material 80 exits the reservoir 34, the reservoir piston 82 can descend, as shown by arrow 86, within the reservoir 34. The descent of the reservoir piston 82 can minimize backpressure that can build within the reservoir 34 as the flowable material 80 exits. The reservoir 34 can be any size without impacting the dose accuracy.

The outlet port 10 can have a Leur taper, Leur lock, or other locking and receiving configurations. The reservoir valve 37 and the outlet valve 43 can be oriented in the same direction (i.e., to allow flow in the same direction along the fluid circuit and to block flow in the same direction in the fluid circuit).

The bolus quantity delivery control can be a bolus dial 68. The bolus dial 68 can be manually set to determine the volume of bolus to be delivered. The dosage setting of the bolus dial 68 can control the location of the piston seal 30 at a desired location inside the cylinder 32, and therefore control the volume differential in the cylinder during the cycle of oscillating the piston seal 30 (i.e., that will be delivered when the piston seal 30 pressurizes the cylinder 32). The bolus dial 68 can be set to deliver large or small bolus quantities and/or deliver the quantity quickly or slowly (e.g., over about one minute) and/or a single time or repeatedly resulting in a bolus delivery and/or a basal delivery from the device 2.

FIG. 8b illustrates that the bolus dial 68 can be manually or automatically rotated, as shown by arrow 78a, to set a bolus dose volume quantity. The piston rod 31 can be forced to translate, as shown by arrow 88, via the piston rack 52 and pinion 48 driven by the bolus dial 68. The pinion 48 can be the central axis or axle of the bolus dial 68. The piston rod 31 can be fixed to the piston seal 30, outlet valve 43, and outlet port 10. The piston seal 30 can have a fluid-tight seal against the inner walls of the cylinder 32. The translating of the piston rod 31 can translate the piston seal 30, outlet valve 43, and outlet port 10, which can create suction or a vacuum that can pull the flowable material 80, as shown by arrow 90, from the reservoir 34. The reservoir valve 37 can allow the flow of flowable material 80 moving in the direction from the reservoir 34 to the cylinder 32, and prevent flow moving in the direction from the cylinder 32 to the reservoir 34. The outlet valve 43 moves with the piston assembly 29 and can prevent the flow of fluid from outside of the outlet port 10 toward the cylinder 32 when the pressure in the cylinder is lower than the pressure outside of the outlet port 10 (e.g., when the piston seal 10 is moving toward the outlet port, as shown by arrow 88).

FIG. 8c illustrates that the bolus dial 68 can then be rotated, as shown by arrow 78b, to return to the original orientation (as shown in FIG. 8a) of the bolus dial 68 and deliver the bolus of fluid in the cylinder 32 shown in FIG. 8b to the outlet port 10. When the dial 68 is manually or automatically rotated back to the original position, as shown by arrow 78b, the piston seal 30 is translated, as shown by arrow 91, (due to the translation of the piston rod 31 which is driven by the piston rack 52 and pinion 48) increasing the fluid pressure in the cylinder 32 and forcing the bolus dosage through the outlet valve 43, out the outlet port 10 and into patient. The outlet valve 43 can allow the flow of flowable material 80, as shown by arrow 92, through the piston fluid channel 33, the outlet valve 43 and out the outlet port 10. The outlet valve 43 can prevent flow from outside of the outlet port toward the cylinder 32. The reservoir valve 37 can close when the cylinder 32 is under positive pressure, preventing the flow of fluid in the cylinder 32 from flowing to the reservoir 34.

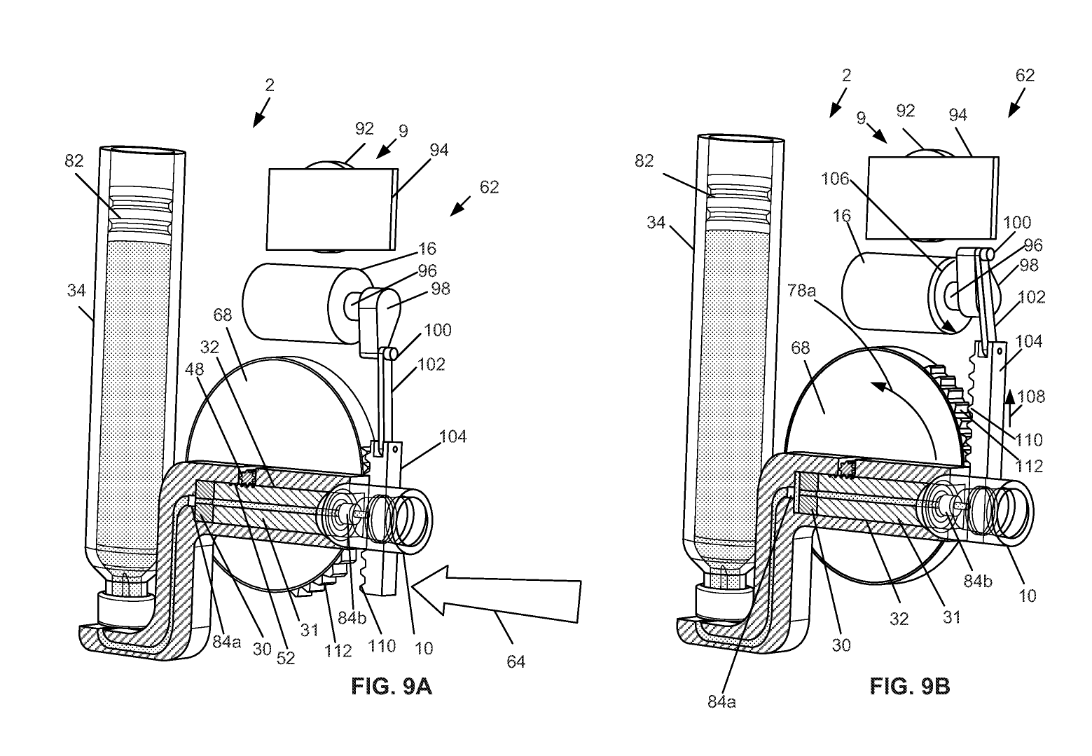

FIG. 9a illustrates that a power pack 62 can be attached, as shown by arrow 64, to drive the bolus dial 68. The power pack 62 can have a battery 92, board 94 (e.g., motherboard, processor, memory), the motor 16, a series of linkages connecting the motor 16 to the bolus dial 68, and combinations thereof. The series of linkages can include a motor shaft 96 hinged to a cam-shaped crank 98 that can be hinged at a cam hinge 100 to a motor link 102 that can be hinged at a distal end to a motor rack 104. The board 94 can control the basal and/or bolus delivery of flowing material 80 out of the device 2 and to the patient. The battery 92 can power the board 94 and the motor 16.

A gear train can be between the motor 16 and the motor link 102. For example, the gear train can control motor rack 104 force, travel distance, delivery rates, or combinations thereof.

The motor rack 104 can have motor rack teeth 110. The bolus dial 68 can have bolus dial teeth 112 along at least a portion of the circumference of the bolus dial 68 that can come into contact with the motor rack 104. The motor rack teeth 110 can be along a length of the motor rack 104 that can contact the bolus dial 68. The motor rack teeth 110 can engage the bolus dial teeth 112. The bolus dial 68 is constrained against the motor rack 104. The board 94 can control the motor 16 to rotate at set rpm to force the motor rack 104 up and down at a desired pace for basal delivery.

FIG. 9b illustrates that rotation of the motor 16, resulting in rotation of the motor shaft 96, as shown by arrow 106, which in turn rotates the crank 98 and rotates and translates the motor link 102. Translation of the motor link 102 in turn translates the motor rack 104, as shown by arrow 108. Translation of the motor rack 104 rotates the bolus dial 68, as shown by arrow 78a. Continued rotation of the motor 16 can result in oscillating motion of the bolus dial 68. The bolus dial 68 can then force the piston back and forth in the cylinder 32, thereby delivering a basal rate of flowable material (e.g., medication) repeatedly and/or continuously.

The motor rack 104 can be left engaged or disengaged from the bolus dial 68 before performing manually controlling the bolus dial 68 to deliver a bolus.

FIG. 10 illustrates that the flowable material delivery mechanism of the device 2 can have the motor 16 connected through a gear and/or shaft 96 to the drive wheel 20. The drive wheel 20 can push the piston 30 via the control tooth 24 and piston cuff 28 (as described supra). The piston cuff 28 can be an abutment. The piston 30 can draw flowable material from the reservoir 34 into the cylinder 32. When the control tooth 24 passes the piston cuff 28, the piston spring 38 can apply pressure against the piston rod 31, which can be fixed to the piston seal 30. The piston spring 38 can be constrained between the housing 40 and the piston cuff 28. The piston seal 30 can then pressurize the flowable material in the cylinder 32, closing the reservoir valve (not shown in FIG. 10) between the cylinder 32 and the reservoir 34, and opening the outlet valve between the cylinder 32 and the outlet port (not shown in FIG. 10). A catheter or other infusion tubing 66 can be attached to the outlet port 10. The flowable material 80 can flow out of the cylinder 32, and through the outlet port 10 and catheter or infusion tubing 66. The catheter or infusion tubing 66 can deliver the flowable material to tissue or secondary devices (e.g., implants) away from the outlet port 10.

The motor 16 shown in FIG. 10 can rotate in a single direction during use.

FIG. 11 illustrates the method performed by the device 2 of FIG. 10. The motor 16 can rotate the drive gear 20. The drive gear 20 can then translate or force the piston 30 away from the reservoir (in the fluid circuit) via single control tooth 24, thereby drawing a dosage of the flowable material into the cylinder 32. When the single control tooth 24 rotates away from piston cuff 28, the piston spring 38 can force the piston seal 30 back to the piston seal's original position, and pushes dosage through outlet port 10 and infusion catheter or other tubing 66. The device 2 can have two valves 37 and 43 to insure proper fluid flow during dosage setting and delivery.

FIG. 12a illustrates that the device can be positioned onto the surface of a patient's skin 200. The case bottom surface 6 can be flat or contoured to match the skin surface. The device 2 can be placed on the skin 200 at or near the abdomen, buttocks, on, attached to or in a belt, bra strap, let strap, or combinations thereof.

The case bottom surface 6 can be coated with an adhesive. The outlet port 10 and/or outlet needle 12 can be at or adjacent a target site 202. The target site 202 can be tissue, such as a muscle, blood vessel, lymph vessel, lymph node, or combinations thereof. The target site 202 can be a device, such as an implanted fluid distribution reservoir, such as a bladder connected to a pump. Basal delivery, as shown by arrows 204, of a flowable material can pass through the end of the outlet needle 12 and to the target site 202.

FIG. 12b illustrates that the reservoir 34 can be filled, including refilling. Although the filling is shown while the delivery of flowable material is occuring, as shown by arrow 204, the device 2 can be filled while delivering fluid or not deliverying fluid. The device 2 can be refilled while on the skin 200, away from the body, or positioned subcutaneously (as shown in FIG. 12f) or transcutaneously. A filling device 206 can have a filling needle 208, a filling syringe 210 that can be partially or completely filled with a flowable material for delivery into the patient. The syringe 210 can have a plunger 212. The refilling needle 208 can be inserted, as shown by arrow 214, into or through the outlet port 8. The plunger 212 can be depressed, as shown by arrow 216. The piston in the refilling syringe can then force the flowable material from the cylinder to the reservoir 34.

FIG. 12c illustrates that the device 2 can deliver a bolus discharge 218 to the target site. The bolus button 14 can be pressed before the bolus discharge.

FIG. 12d illustrates that the outlet port 10 of the device 2 can be attached to a transcutaneous delivery apparatus 220. The transcutaneous delivery apparatus 220 can be configured to aid in the transmission of matter through skin. For example, the transcutaneous delivery apparatus 220 can be a dermal patch, an iontophoresis device, an electroporation device, or combinations thereof.

FIG. 12e illustrates that a catheter or catheter assembly 66 can extend from the outlet port 10. The catheter assembly 66 can include tubing (e.g., the internal outlet connector) and a catheter configured to deliver the flowable material to a remote anatomical target site. The catheter assembly 66 can extend distally from the outlet port 6. The catheter assembly 66 can deliver the flowable material out of the distal port of the terminal distal end of the catheter and/or through perforations of the lateral sides of the catheter.

FIG. 12f illustrates that the device 2 can be placed entirely (as shown) or partially subcutaneously or viscerally. The bolus button 14 can be on the top surface of the case 4, or the surface of the case 4 closest to the skin 200. The bolus button 14 can be detected and pressed through the skin 200. The reservoir can be refilled by injection of flowable material through the intake port 8.

FIG. 12g illustrates that the device 2 can be away from the skin 200, for example on a belt or clipped or otherwise secured to a piece of clothing. The outer port 10 can be attach to a flexible extension tube 222 that can be attached to or part of the internal outlet connector 45. The extension tube 222 can be attached, for example with a releasable and lockable connector 224, at a location outside of the body (as shown), at the skin 200 or subcutaneously to the catheter 66.

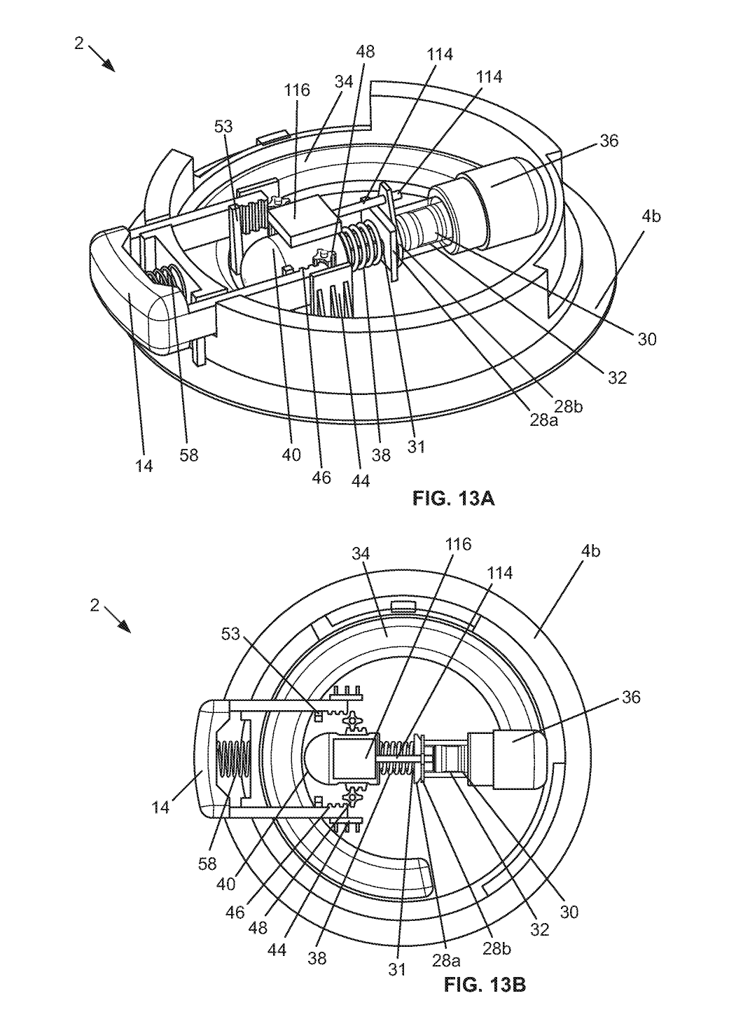

FIGS. 13a and 13b illustrate that the device can have a shape memory cord or wire 114. The shape memory wire 114 can be made from a shape memory material, such as a shape memory alloy (e.g., a nickel titanium alloy, such as Nitinol) or a shape memory polymer.

The device 2 can have an electronics board 116 fixed to the housing 40 or case 4 and to a distal end of the shape memory wire 114. The electronics board 116 can have a power supply, a processor and memory. The electronics board 116 can be configured to control an electrical current flow and/or heat and cooling delivered to the shape memory wire 114.

The piston assembly 29 can have a first piston cuff 28a and a second piston cuff 28b. The second piston cuff 28b can extend laterally beyond the first piston cuff 28a (as shown in FIG. 13a). The piston spring 38 can be constrained between the first piston cuff 28a and the housing 40. The second piston cuff 28b can be attached to the wire 114.

The electronics board 116 can control the length of the shape memory wire 114, for example by delivering an increasing or decreasing amount of electrical current or heat or cooling. The electronics board 116 can oscillate the length of the shape memory wire 114, for example to control a basal delivery of the flowable material in the reservoir 34 through the outlet valve 10 and to the patient.

The device 2, such as the case 4 or any other components, can be made from polycarbonate, ABS, polypropolene, polyeurethane, nylon, rubber, aluminum, magnesium, or any combination thereof.

Currently pending U.S. application Ser. No. 12/246,230, filed 6 Oct. 2008, is incorporated by reference herein in its entirety.

The delivering of the basal infusion can include delivering fluid continuously for an extended period of time defined by the type of medication or other agents within the fluid. The delivering of the bolus can include delivering fluid for single doses over short periods of time defined by the type of medication or other agents within the fluid.

For example, bolus insulin delivery volumes can range from about 1 U (e.g., 0.01 mL of insulin) to about 10 U (e.g., 0.1 mL of insulin). Bolus delivery can be completed, as measured from the beginning to the end of the active delivery of the fluid, in less than about 1 minute, more narrowly in less than about 15 seconds, yet more narrowly in less than about 5 seconds. Basal delivery rates range from about 0.1 U per hour (e.g., 0.001 mL/hr of insulin) to about 4 U per hour (e.g., 0.04 mL/hr of insulin). Basal delivery can be completed, as measured from the beginning to the end of the active delivery of the fluid, in more than about 1 minute, more narrowly more than or equal to about 5 minutes, yet more narrowly more than or equal to about 1 hour, yet more narrowly more than or equal to about 3 hours, yet more narrowly more than or equal to about 5 hours, yet more narrowly more than or equal to about 8 hours.

Bolus delivery can include single strokes of the piston assembly 29 that deliver a volume of equal to or more than about 0.2 U (e.g., 0.002 mL of insulin) or greater. Basal delivery can include single strokes of the piston assembly 29 that deliver a volume of less than about 0.2 U (e.g., 0.002 mL of insulin).

1 mL of insulin contains 100 Units (U) of insulin. 1 mL of another medication can have a different number of units (e.g., herapin has 10,000 U per 1 mL).

The flowable material can include any of the following in solid, liquid, gel, or forms that are a combination thereof: natural and/or synthetic insulin or insulin analogues (e.g., extended release/protracted insulin, rapid acting/fast acting insulin, insulin lispro, NovoRapid, Apidra, or combinations thereof); heparin; myristyric acid; serum albumin; antineoplastic agents; alkylating agents; anti-metabolites; plant alkaloids; terpenoids; vinca alkaloids; podophyllotoxin; taxanes; topoisomerase inhibitors; radioactive materials; radiopaque materials; cytogenic agents; cytotoxic agents; cytostatic agents; thrombogenic agents, for example polyurethane, cellulose acetate polymer mixed with bismuth trioxide, and ethylene vinyl alcohol; lubricious, hydrophilic materials; phosphor cholene; anti-inflammatory agents, for example non-steroidal anti-inflammatories (NSAIDs) such as cyclooxygenase-1 (COX-1) inhibitors (e.g., acetylsalicylic acid, for example ASPIRIN® from Bayer AG, Leverkusen, Germany; ibuprofen, for example ADVIL® from Wyeth, Collegeville, Pa.; indomethacin; mefenamic acid), COX-2 inhibitors (e.g., VIOXX® from Merck & Co., Inc., Whitehouse Station, N.J.; CELEBREX® from Pharmacia Corp., Peapack, N.J.; COX-1 inhibitors); immunosuppressive agents, for example Sirolimus (RAPAMUNE®, from Wyeth, Collegeville, Pa.), or matrix metalloproteinase (MMP) inhibitors (e.g., tetracycline and tetracycline derivatives) that act early within the pathways of an inflammatory response. Examples of other agents are provided in Walton et al, Inhibition of Prostoglandin E₂ Synthesis in Abdominal Aortic Aneurysms, Circulation, Jul. 6, 1999, 48-54; Tambiah et al, Provocation of Experimental Aortic Inflammation Mediators and Chlamydia Pneumoniae, Brit. J. Surgery 88 (7), 935-940; Franklin et al, Uptake of Tetracycline by Aortic Aneurysm Wall and Its Effect on Inflammation and Proteolysis, Brit. J. Surgery 86 (6), 771-775; Xu et al, Spl Increases Expression of Cyclooxygenase-2 in Hypoxic Vascular Endothelium, J. Biological Chemistry 275 (32) 24583-24589; and Pyo et al, Targeted Gene Disruption of Matrix Metalloproteinase-9 (Gelatinase B) Suppresses Development of Experimental Abdominal Aortic Aneurysms, J. Clinical Investigation 105 (11), 1641-1649 which are all incorporated by reference in their entireties.

It is apparent to one skilled in the art that various changes and modifications can be made to this disclosure, and equivalents employed, without departing from the spirit and scope of the invention. Elements expressed herein as singular or plural can be used in the alternative (i.e., singular as plural and plural as singular). Elements shown with any embodiment are exemplary for the specific embodiment and can be used in combination on or with other embodiments within this disclosure.

We claim:

1. A device for delivering a fluid to a biological tissue comprising:
a reservoir containing the fluid and having a fluid pressure;
a basal control configured to change the fluid pressure in the reservoir;
a bolus control configured to change the fluid pressure in the reservoir;
a pressure control element in fluid communication with the reservoir, wherein the basal control is configured to control the pressure control element, and wherein the bolus control is configured to control the pressure control element; and
an outlet port on the device configured to provide access from the pressure control element to the tissue.

2. The device of claim 1, wherein the bolus control is mechanically independent from the basal control.

2. The device of claim 1, wherein the pressure control element comprises a piston.

3. The device of claim 1, wherein the pressure control element comprises a one-way valve.

4. The device of claim 1, further comprising a first valve between the pressure control element and the reservoir, and a second valve between the pressure control element and the outlet port, wherein the first valve comprises a one-way valve configured to allow flow out of the reservoir and to inhibit flow into the reservoir, wherein the second valve comprises a one-way valve configured to allow flow from the pressure control element to the outlet port and to inhibit flow from the outlet port toward the pressure control element.

5. The device of claim 1, further comprising a first one-way valve and a second one-way valve, wherein the first one-way valve and the second one-way valve allow flow in a first direction and inhibit flow in a second direction, and wherein the second one way valve is in fluid communication with the outlet port and the pressure control element.

6. The device of claim 1, wherein the basal control comprises a shape memory metal.

7. The device of claim 1, wherein the bolus control comprises a handle and/or button, and wherein the bolus control is configured to be exclusively manually triggered.

8. The device of claim 1, wherein the basal control comprises an electric control.

9. The device of claim 8, wherein the electric control comprises a motor and a worm gear.

10. The device of claim 1, wherein the reservoir is flexible.

11. The device of claim 1, further comprising a tube attached to the outlet port.

12. The device of claim 1, further comprising a catheter attached to the outlet port.

13. The device of claim 1, wherein the pressure control element comprises a fluid channel through the pressure control element configured to allow the fluid to flow through the pressure control element.

14. A device for delivering a fluid to a biological tissue comprising:
a flexible reservoir containing the fluid and having a fluid pressure;
a pressure control element in fluid communication with the reservoir, wherein the pressure control element is configured to controllably alter the fluid pressure in the reservoir;
an electric control configured to control the pressure control element; and
a manual control configured to control the pressure control element.

15. A method for delivering a fluid to a biological tissue comprising:
delivering a basal infusion of the fluid from a reservoir of a fluid delivery device, wherein the delivering of the basal infusion comprises a first changing of the pressure of the reservoir, and wherein the first changing of the pressure comprises moving a pressure control element with an electric motor; and
delivering a bolus of the fluid from the fluid delivery device, wherein the delivering of the bolus of the fluid comprises a second changing of the pressure of the reservoir, and wherein the second changing of the pressure comprises moving the pressure control element with a manual force.

16. The method of claim 15, further comprising stopping the delivering of the basal infusion of the fluid before delivering the bolus of the fluid.

17. The method of claim 15, wherein the delivering of the basal infusion further comprises moving the pressure control element with a spring after the first changing of the pressure of the reservoir.

18. The method of claim 17, wherein the delivering of the bolus infusion further comprises moving the pressure control element with the spring after the second changing of the pressure of the reservoir

19. The method of claim 16, further comprising removing the basal control before the the delivering of the bolus of fluid.

20. The method of claim 16, further comprising adjusting a volume of the bolus of the fluid before delivering the bolus of the fluid, wherein adjusting comprises adjusting a limit of the motion of the pressure control element.
